(19) United States
(12) Patent Application Publication
(10) Pub. No.: US 2009/0275935 A1
(43) Pub. Date: Nov. 5, 2009

(54) CANNULA ENCLOSING RECESSED WAVEGUIDE OUTPUT TIP

(76) Inventor: Ronald D. McKee, Lake Forest, CA (US)

Correspondence Address: STOUT, UXA, BUYAN & MULLINS LLP, 4 VENTURE, SUITE 300, IRVINE, CA, 92618, US

(21) Appl. No.: 12/434,460

(22) Filed: May 1, 2009

Related U.S. Application Data
(60) Provisional application No. 61/049,544, filed on May 1, 2008.

(51) Int. Cl. A61B 18/18

(52) U.S. Cl. 606/16

(57) ABSTRACT

A cannula includes a proximal end, a distal end, and a cannula axis extending between the proximal end and the distal end. A lumen is disposed along at least a portion of the cannula between the proximal end and the distal end, and an energy output is configured to ablate a target surface, the energy output having a longitudinal axis and a tissue-disrupting distal end that is disposed within the lumen near the distal end. The tissue-disrupting distal end is configured to direct ablating energy in a direction toward the distal end of the cannula. The cannula axis and the longitudinal axis are concentric, and a region between the tissue-disrupting distal end and the distal end of the cannula is fixed and further can be transparent to a wavelength of energy emitted from the energy output.

### PRIORITY INFORMATION

This application claims the benefit of U.S. Provisional Application 61/049,544, filed May 1, 2008, the contents of which are expressly incorporated herein by reference.

### BACKGROUND OF THE INVENTION

1. Field of the Invention

The present invention relates generally to electromagnetic radiation procedural devices and, more particularly, to the use of electromagnetic radiation devices in medical applications.

2. Description of Related Art

Lasers, such as mid-infrared lasers including the Erbium, chromium:yttrium-scandium gallium-garnet (Er,Cr:YSGG) laser, have been used in various treatment procedures. The Er,Cr:YSGG laser is known to be capable of removing tissues by emitting a beam of infrared energy in combination with an emitted water spray.

A prior art optical cutter can include a fiber guide tube, a water line, an air line, and an air knife line for supplying pressurized air. A cap can be fitted onto the hand-held apparatus and secured via threads. The fiber guide tube abuts within a cylindrical metal piece, and another cylindrical metal piece can also be a part of the cap. The pressurized air from the air knife line surrounds and cools the laser, as the laser bridges a gap between the two metal cylindrical objects.

The laser energy exits from the fiber guide tube and is applied to a target surface of the patient. Water from the water line and pressurized air from the air line are forced into a mixing chamber. The air and water mixture is output onto and travels along the outside of the fiber guide tube, and then leaves the tube and contacts the area of surgery.

One prior art arrangement includes an optical cutting system utilizing the expansion of water to destroy and remove tooth material, such as disclosed in U.S. Pat. No. 5,199,870 to Steiner et al. U.S. Pat. No. 5,267,856 to Wolbarsht et al. discloses a cutting apparatus that irradiates a target with laser energy in the presence of water. In both patents the precision and accuracy of the cut is highly dependent upon the precision and accuracy of the water film on or within pores of the material.

### SUMMARY OF THE INVENTION

The present invention comprises an electromagnetically induced cutter, which can provide accurate cutting operations on hard and soft tissues, and other materials as well. Soft tissues may include fat, skin, mucosa, gingiva, muscle, connective tissue, heart, liver, kidney, brain, eye, and vessels, and hard tissue may include tooth enamel, tooth dentin, tooth cementum, tooth decay, amalgam, composites materials, tarter and calculus, bone and cartilage.

According to one implementation, a cannula includes a proximal end, a distal end, and a cannula axis extending between the proximal end and the distal end. A lumen is disposed along at least a portion of the cannula between the proximal end and the distal end, and an energy output is configured to ablate a target surface, the energy output having a longitudinal axis and a tissue-disrupting distal end that is disposed within the lumen near the distal end. The tissue-disrupting distal end is configured to direct ablating energy in a direction toward the distal end of the cannula. The cannula axis and the longitudinal axis are concentric, and a region between the tissue-disrupting distal end and the distal end of the cannula is fixed and further is transparent to a wavelength of energy emitted from the energy output.

A laser having a high absorption for one or more predetermined fluids, which are disposed either around or adjacent to a target tissue or disposed within the target tissue, can be implemented to achieve, for example, intra-tissue or intra-lumen treatments. The laser can be operated to cut or otherwise treat tissue in a vicinity of a target.

While the apparatus and method has or will be described for the sake of grammatical fluidity with functional explanations, it is to be expressly understood that the claims, unless expressly formulated under 35 USC §112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC §112 are to be accorded full statutory equivalents under 35 USC §112.

Any feature or combination of features described or referenced herein are included within the scope of the present invention provided that the features included in any such combination are not mutually inconsistent as will be apparent from the context, this specification, and the knowledge of one skilled in the art. In addition, any feature or combination of features may be specifically excluded from any embodiment of the present invention. For purposes of summarizing the present invention, certain aspects, advantages and novel features of the present invention are described. Of course, it is to be understood that not necessarily all such aspects, advantages or features will be embodied in any particular implementation of the present invention. Additional advantages and aspects of the present invention are apparent in the following detailed description and additional disclosure in claims format that follow.

### BRIEF DESCRIPTION OF THE DRAWINGS

FIG. 1 is an electromagnetic energy waveguide non-slidably disposed within a cannula optical cutter apparatus according to an embodiment of the present invention;

FIG. 2 is an electromagnetic energy waveguide non-slidably disposed within a cannula optical cutter apparatus according to another embodiment of the present invention; and

FIG. 3 is yet another embodiment of an electromagnetic energy waveguide non-slidably disposed within a cannula optical cutter apparatus according to the present invention.

### DETAILED DESCRIPTION OF THE INVENTION

Reference will now be made to certain embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain embodiments, it is to be understood that these embodiments are presented by way of example and not by way of limitation. The intent of this disclosure, while discussing exemplary embodiments, is that the following detailed description be construed to cover all modifications, alternatives, and equivalents of the embodiments as may fall within the spirit and scope of the invention as defined by the additional disclosure in claims format. It is to be understood and appreciated that the process steps and structures described herein do not cover a complete architecture or process, and only so much of the commonly practiced features and steps are included herein as are necessary to provide an understanding of the present invention.

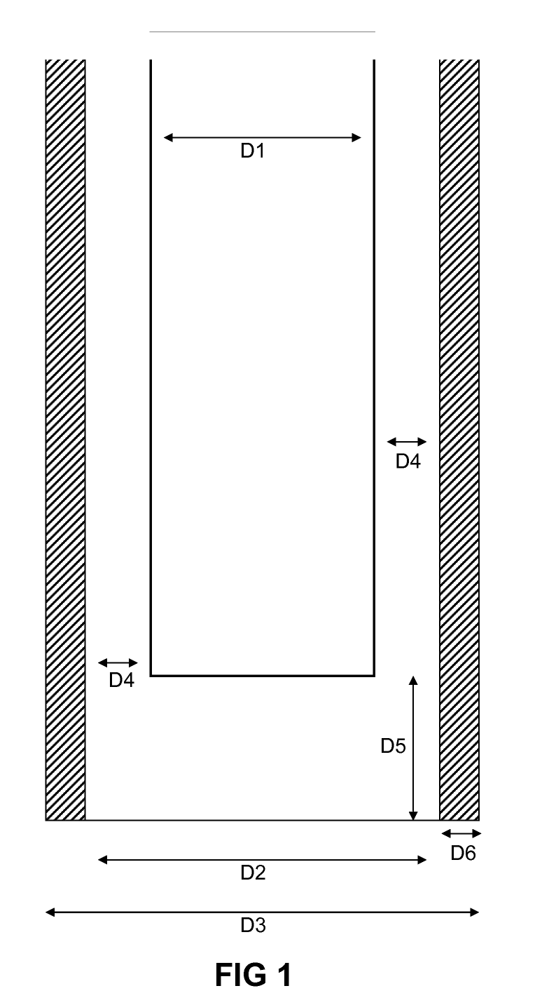

As depicted in the attached FIG. 1, an apparatus can be provided with an electromagnetic energy waveguide that is disposed (i.e., non-slidably disposed) within a cannula for emitting electromagnetic energy from an output end of the electromagnetic energy waveguide and out of the cannula. The electromagnetic energy waveguide as depicted can comprise, in connection with an aspect of the present invention, a fiber optic disposed (e.g., inserted, so that it floats within the cannula lumen) within a cannula (e.g., a stainless steel cannula) for emitting optical (e.g., laser) energy from a distal output end of the fiber optic and out of a distal end of the stainless steel cannula.

An inner diameter D1 of the fiber optic can range from about 200 to 1000 μm, or in certain implementations from about 400 to 600 μm, or in an illustrated embodiment can be about 400 μm. The cannula can have an inner diameter D2 in a range from about 400 mm to 1400 μm, or in certain implementations from about 400 to 1200 μm, or in an illustrated embodiment can be about 600 μm, and the cannula can have an outer diameter D3 in a range from about 600 mm to 1800 μm, or in certain implementations from about 600 to 1600 μm, or in an illustrated embodiment can be about 800 μm.

A gap D4 between an outer surface of the fiber optic and an interior surface of the cannula wall can range from about 50 μm to 500 μm, or in certain implementations from about 100 to 300 μm, or in an illustrated embodiment can be about 200 μm.

While the inner diameter D1 of the fiber optic and the inner diameter D2 of the cannula are, of course, fixed in any given implementation, the gap D4 values are not. In other words, the fiber optic may not be perfectly concentric with the lumen of the cannula at certain orientations or times. Thus, the fiber optic can be allowed to float, so that, according to one embodiment, at a given moment or orientation the fiber optic may not be perfectly centered in the lumen of the cannula, whereby the axes of the fiber optic and the cannula may not be identical or overlapping. In other implementations, structure (e.g., spacers) may be provided to fix the gap D4 values.

A distance D5 at which the output (distal) end of the fiber optic is recessed behind the distal end of the cannula is fixed. The gap can, for example, be set to optimize a cutting efficiency, and/or can be set in a range from about 500 mm to 3000 μm, or in certain implementations from about 100 to 2000 μm, or in an illustrated embodiment can be about 1000 μm or 1500 μm. Finally, a wall thickness D6 of the cannula can range, for example, from about 100 to 200 μm,

According to one broad aspect, the cannula and waveguide combination can be operated in a contact-tip mode of operation to perform one or more of contacting the target surface and effectuating treatment (e.g., ablation) of a portion of the target surface.

The electromagnetic energy may comprise laser energy and/or visible light and may operate to provide or promote one or more of desterilization, bacterial reduction, biostimulation (e.g., low-level light therapy), coagulation, remodeling, caries detection or treatment, and illumination (e.g., with visible light).

In certain implementations, the electromagnetic energy can comprise one or more of an electromagnetic energy source of ablation, and/or an electromagnetic energy source of illumination, and/or an electromagnetic energy source of tissue disruption, and/or an electromagnetic energy source of biostimulation.

The target surface may comprise, for example, one or more of tooth tissue, bone, cartilage and soft tissue such as skin or nasal-cavity tissue.

According to certain aspects of the present invention, the energy output can comprise one or more of hard-tissue ablating electromagnetic energy, low-level light therapy (LLLT) electromagnetic energy, tissue-biostimulation electromagnetic energy, visible electromagnetic energy, coherent light, one or more of a wavelength within a range from about 2.69 to about 2.80 microns and a wavelength of about 2.94 microns, and electromagnetic energy generated by one or more of an Er:YAG laser, an Er:YSGG laser, an Er, a Cr:YSGG laser and a CTE:YAG laser.

In one implementation, the cannula can be configured to direct liquid in a direction toward the distal end of the cannula. For example, a fluid can be routed distally along an outer surface (e.g., the entire or substantially the entire outer surface, near the distal end) of the cannula and/or can be routed distally to the distal end of the cannula with one or more fluid delivery conduits or passages, and/or can be supplied from a source other than the distal end of the cannula (e.g., so that the fluid is emitted to travel distally along a path that is not exactly parallel to an axis of the electromagnetic energy waveguide).

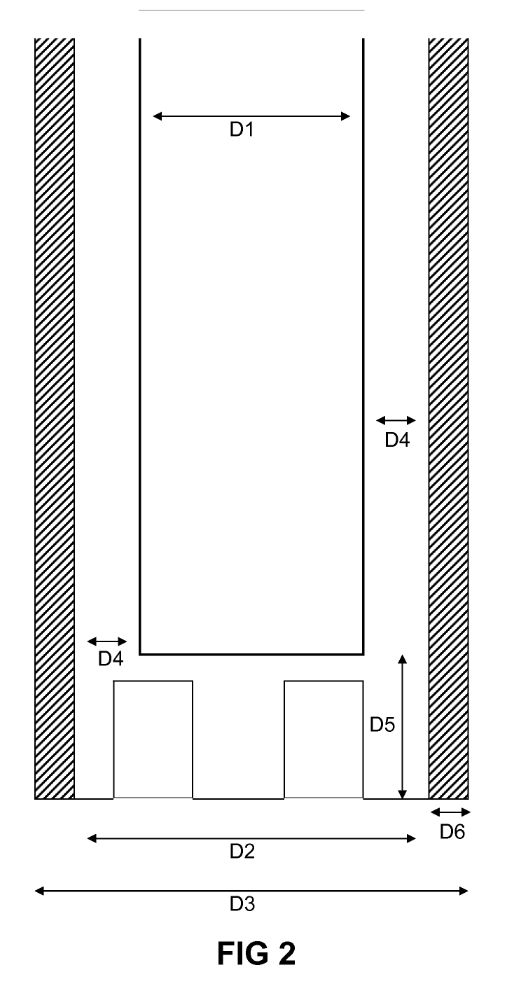

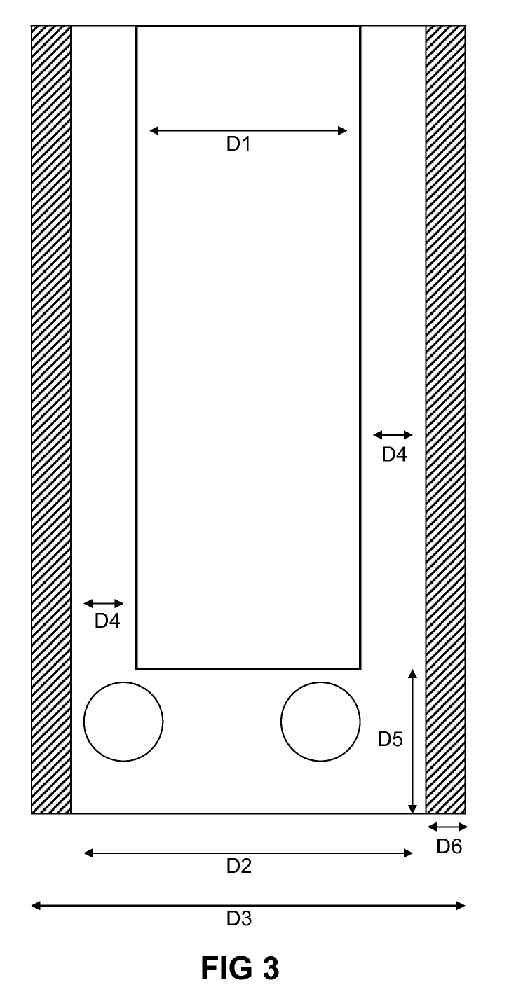

In another implementation, fluid may be supplied through one or more gaps disposed between the outer surface of the waveguide (e.g., fiber optic) and the interior surface of the cannula. The fluid can be a liquid or may comprise a combination of liquid and gas. In certain implementations, the liquid is or comprises water, and in other implementations it is or comprises both air and water which, for example, can be mixed together either before or within the gap. For example, the fluid can comprise atomized fluid particles formed from a mixture of pressurized air and water and delivered through the gap to exit from the fluid output. At least one pressure output port can be disposed at the distal end of the cannula, as depicted in FIGS. 2 and 3.

When the apparatus is operated in a contact-tip mode, or not, the volume between the tissue ablating and/or tissue-treating distal end and the distal end of the cannula can be transparent to a wavelength of energy emitted from the energy output. According to another implementation, in addition to or as an alternative to the features of the preceding sentence, when the apparatus is operated in a contact-tip mode, or not, a volume between (a) the tissue ablating and/or tissue-treating distal end and (b) the distal end of the cannula does not obstruct atomized fluid particles traveling in the direction from the fluid output to the distal end of the cannula. According to yet another implementation, in addition to or as an alternative to any one or more features set forth in this paragraph, when the apparatus is operated in a contact-tip mode, or not, a volume between (a) the tissue ablating and/or tissue-treating distal end and (b) the target surface is not obstructed by any part of the apparatus.

According to other implementations, the apparatus can comprise a fluid output that is configured to emit fluid in a vicinity of the distal end of the apparatus, wherein: the fluid output comprises an atomizer configured to place atomized fluid particles into a volume above the target surface; and the electromagnetic energy waveguide is configured to impart relatively large amounts of energy into the atomized fluid particles in the volume above the target surface to thereby expand the atomized fluid particles and impart disruptive forces onto the target surface.

Corresponding or related structure and methods described in the following patents assigned to Biolase Technology, Inc., are incorporated herein by reference in their entireties, wherein such incorporation includes corresponding or related structure (and modifications thereof) in the following patents which may be, in whole or in part, (i) operable with, (ii) modified by one skilled in the art to be operable with, and/or (iii) implemented/used with or in combination with, any part(s) of the present invention according to this disclosure, that of the patents or below applications, and the knowledge and judgment of one skilled in the art:
U.S. Patent No. Title
U.S. Pat. No. 7,356,208 Fiber detector apparatus and related methods
U.S. Pat. No. 7,320,594 Fluid and laser system
U.S. Pat. No. 7,303,397 Caries detection using timing differentials between excitation and return pulses
U.S. Pat. No. 7,292,759 Contra-angle rotating handpiece having tactile-feedback tip ferrule
U.S. Pat. No. 7,290,940 Fiber tip detector apparatus and related methods
U.S. Pat. No. 7.288.086 High-efficiency, side-pumped diode laser system
U.S. Pat. No. 7,270,657 Radiation emitting apparatus with spatially controllable output energy distributions
U.S. Pat. No. 7,261,558 Electromagnetic radiation emitting toothbrush and dentifrice system
U.S. Pat. No. 7,194,180 Fiber detector apparatus and related methods
U.S. Pat. No. 7,187,822 Fiber tip fluid output device
U.S. Pat. No. 7,144,249 Device for dental care and whitening
U.S. Pat. No. 7,108,693 Electromagnetic energy distributions for electromagnetically induced mechanical cutting
U.S. Pat. No. 7,068,912 Fiber detector apparatus and related methods
U.S. Pat. No. 6,942,658 Radiation emitting apparatus with spatially controllable output energy distributions
U.S. Pat. No. 6,829,427 Fiber detector apparatus and related methods
U.S. Pat. No. 6,821,272 Electromagnetic energy distributions for electromagnetically induced cutting
U.S. Pat. No. 6,744,790 Device for reduction of thermal lensing
U.S. Pat. No. 6,669,685 Tissue remover and method
U.S. Pat. No. 6,616,451 Electromagnetic radiation emitting toothbrush and dentifrice system
U.S. Pat. No. 6,616,447 Device for dental care and whitening
U.S. Pat. No. 6,610,053 Methods of using atomized particles for electromagnetically induced cutting
U.S. Pat. No. 6,567,582 Fiber tip fluid output device
U.S. Pat. No. 6,561,803 Fluid conditioning system
U.S. Pat. No. 6,544,256 Electromagnetically induced cutting with atomized fluid particles for dermatological applications
U.S. Pat. No. 6,533,775 Light-activated hair treatment and removal device
U.S. Pat. No. 6,389,193 Rotating handpiece
U.S. Pat. No. 6,350,123 Fluid conditioning system
U.S. Pat. No. 6,288,499 Electromagnetic energy distributions for electromagnetically induced mechanical cutting
U.S. Pat. No. 6,254,597 Tissue remover and method
U.S. Pat. No. 6,231,567 Material remover and method
U.S. Pat. No. 6,086,367 Dental and medical procedures employing laser radiation
U.S. Pat. No. 5,968,037 User programmable combination of atomized particles for electromagnetically induced cutting
U.S. Pat. No. 5,785,521 Fluid conditioning system
U.S. Pat. No. 5,741,247 Atomized fluid particles for electromagnetically induced cutting

Also, the above disclosure and referenced items are intended to be operable or modifiable to be operable, in whole or in part, with corresponding or related structure and methods, in whole or in part, described in the following applications and items referenced therein, which applications include U.S. application Ser. No. 12/234,593, filed Sep. 19, 2008 and entitled PROBES AND BIOFLUIDS FOR TREATING AND REMOVING DEPOSITS FROM TISSUE SURFACES (Docket BI8053P) and U.S. application Ser. No. 10/667,921, filed Sep. 22, 2003 and entitled TISSUE REMOVER AND METHOD (Docket BI9100CIPCON), and which applications are listed as follows:
U.S. App. Pub. No. Title
20080070185 Caries detection using timing differentials between excitation and return pulses
20080065057 High-efficiency, side-pumped diode laser system
20080065055 Methods for treating eye conditions
20080065054 Methods for treating hyperopia and presbyopia via laser tunneling
20080065053 Methods for treating eye conditions
20080033411 High efficiency electromagnetic laser energy cutting device
20080033409 Methods for treating eye conditions
20080033407 Methods for treating eye conditions
20080025675 Fiber tip detector apparatus and related methods
20080025672 Contra-angle rotating handpiece having tactile-feedback tip ferrule
20080025671 Contra-angle rotating handpiece having tactile-feedback tip ferrule
20070298369 Electromagnetic radiation emitting toothbrush and dentifrice system
20070263975 Modified-output fiber optic tips
20070258693 Fiber detector apparatus and related methods
20070208404 Tissue treatment device and method
20070208328 Contra-angel rotating handpiece having tactile-feedback tip ferrule
20070190482 Fluid conditioning system
20070184402 Caries detection using real-time imaging and multiple excitation frequencies
20070104419 Fiber tip fluid output device
20070060917 High-efficiency, side-pumped diode laser system
20070059660 Device for dental care and whitening
20070054236 Device for dental care and whitening
20070054235 Device for dental care and whitening
20070054233 Device for dental care and whitening
20070042315 Visual feedback implements for electromagnetic energy output devices
20070014517 Electromagnetic energy emitting device with increased spot size
20070014322 Electromagnetic energy distributions for electromagnetically induced mechanical cutting
20070009856 Device having activated textured surfaces for treating oral tissue
20070003604 Tissue coverings bearing customized tissue images
20060281042 Electromagnetic radiation emitting toothbrush and dentifrice system
20060275016 Contra-angle rotating handpiece having tactile-feedback tip ferrule
20060241574 Electromagnetic energy distributions for electromagnetically induced disruptive cutting
20060240381 Fluid conditioning system
20060210228 Fiber detector apparatus and related methods
20060204203 Radiation emitting apparatus with spatially controllable output energy distributions
20060142743 Medical laser having controlled-temperature and sterilized fluid output
20060099548 Caries detection using timing differentials between excitation and return pulses
20060043903 Electromagnetic energy distributions for electromagnetically induced mechanical cutting
20050283143 Tissue remover and method
20050281887 Fluid conditioning system
20050281530 Modified-output fiber optic tips
20040106082 Device for dental care and whitening
20040092925 Methods of using atomized particles for electromagnetically induced cutting
20040091834 Electromagnetic radiation emitting toothbrush and dentifrice system
20040068256 Tissue remover and method
20030228094 Fiber tip fluid output device
20020149324 Electromagnetic energy distributions for electromagnetically induced mechanical cutting
20020014855 Electromagnetic energy distributions for electromagnetically induced mechanical cutting

All of the contents of the preceding published applications are incorporated herein by reference in their entireties.

The above-described embodiments have been provided by way of example, and the present invention is not limited to these examples. Multiple variations and modifications to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. As iterated above, any feature or combination of features described and referenced herein are included within the scope of the present invention provided that the features included in any such combination are not mutually inconsistent as will be apparent from the context, this specification, and the knowledge of one of ordinary skill in the art. For example, any of the mentioned elements, and other components, and any particulars or features thereof, or other features, including method steps and techniques, may be used with any other structure and process described or referenced herein, in whole or in part, in any combination or permutation, as a non-equivalent, separate and non-interchangeable aspect of this application. Accordingly, the present invention is not intended to be limited by the disclosed embodiments, but is to be defined by such embodiments and by reference to the following additional disclosure in claims format.

What is claimed is:

1. A cannula comprising:
a proximal end, a distal end, and a cannula axis extending between the proximal end and the distal end;
a lumen disposed along at least a portion of the cannula between the proximal end and the distal end; and
an energy output configured to ablate a target surface, the energy output having a longitudinal axis and a tissue-disrupting distal end that is disposed within the lumen near the distal end, the tissue-disrupting distal end being configured to direct ablating energy in a direction toward the distal end of the cannula;
wherein the cannula axis and the longitudinal axis are about concentric and a region between the tissue-disrupting distal end and the distal end of the cannula is fixed and further is transparent to a wavelength of energy emitted from the energy output.

2. The cannula as set forth in claim 1, wherein the target surface is skin.

3. The cannula as set forth in claim 1, wherein the energy output is a fiber optic.

4. The cannula as set forth in claim 1, wherein the energy output is configured to emit electromagnetic energy, which comprises soft-tissue ablating electromagnetic energy.

5. The cannula as set forth in claim 1, wherein the energy output is configured to emit electromagnetic energy, which comprises low-level light therapy (LLLT) electromagnetic energy.

6. The cannula as set forth in claim 1, wherein the energy output is configured to emit electromagnetic energy, which comprises tissue-biostimulation electromagnetic energy.

7. The cannula as set forth in claim 1, wherein the energy output is configured to emit electromagnetic energy, which comprises visible electromagnetic energy.

8. The cannula as set forth in claim 1, wherein the energy output is configured to emit electromagnetic energy, which comprises coherent light.

9. The cannula as set forth in claim 1, and further comprising a fluid output configured to emit fluid in a vicinity of the distal end of the cannula.

10. The cannula as set forth in claim 9, wherein the fluid output is configured to direct liquid in a direction toward the tissue-disrupting distal end.

11. The cannula as set forth in claim 9, wherein the fluid is water.

12. The apparatus as set forth in claim 11, wherein the energy output is configured to emit electromagnetic energy, which comprises one of a wavelength within a range from about 2.69 to about 2.80 microns and a wavelength of about 2.94 microns.

13. The apparatus as set forth in claim 11, wherein the energy output is configured to emit electromagnetic energy, which is generated by one or more of an Er:YAG, an Er:YSGG, an Er, Cr:YSGG and a CTE:YAG laser.

14. The apparatus as set forth in claim 11, wherein the apparatus is a nasal-cavity operating apparatus and the target surface is nasal-cavity tissue.

15. The apparatus as set forth in claim 11, wherein the target surface comprises one or more of bone, cartilage and soft tissue.

16. The apparatus as set forth in claim 1, and further comprising a fluid output configured to emit fluid in a vicinity of the distal end of the cannula, wherein:
the fluid output comprises an atomizer configured to place atomized fluid particles into a volume above the target surface; and
the energy output is configured to impart relatively large amounts of energy into the atomized fluid particles in the volume above the target surface to thereby expand the atomized fluid particles and impart the disruptive forces onto the target surface.

17. The apparatus as set forth in claim 16, wherein the fluid particles comprise water.

18. A cannula comprising a proximal end, a distal end, a cannula axis extending between the proximal end and the distal end, and a lumen existing along at least a portion of the cannula between the proximal end and the distal end, the cannula comprising an energy waveguide that has a waveguide axis that is disposed along the cannula axis and that is configured to treat a target surface, the energy output having a tissue-treating distal end positioned within the lumen near but not at the distal end, the tissue-treating distal end being configured to direct treatment energy in a direction toward the distal end of the cannula, and a volume between the tissue-treating distal end and the distal end of the cannula being unchangeable and unobstructed to a wavelength of energy emitted from the energy output.

19. The apparatus as set forth in claim 18, wherein the target surface is skin.

20. The cannula as set forth in claim 18, wherein the source of tissue displacement is a fiber optic.
